(19) United States
(12) Patent Application Publication
(10) Pub. No.: US 2011/0201634 A1
(43) Pub. Date: Aug. 18, 2011

(54) DIHYDROPYRIDIMIDINONE COMPOUNDS FOR THE TREATMENT OF CARDIOVASCULAR DISEASES AND PROCESS FOR PREPARING THE SAME

(75) Inventors: Virinder Singh Parmar, Delhi (IN); Hanumanthrao Guru Raj, Delhi (IN); Ashok Kumar Prasad, Delhi (IN)

(73) Assignees: DELHI UNIVERSITY, Delhi (IN); VALLABHBHAI PATEL CHEST INSTITUTE, Delhi (IN)

(21) Appl. No.: 13/057,247

(22) PCT Filed: Jun. 15, 2009
(86) PCT No.: PCT/IN2009/000344

§ 371 Date: May 2, 2011

(30) Foreign Application Priority Data
Jun. 13, 2008 (IN) 1414/DEL/2008

(51) Int. Cl. A61K 31/513; C07D 239/22; A61P 9/00; A61P 7/02

(52) U.S. Cl. 514/274; 544/318; 544/316

(57) ABSTRACT

The present invention relates to a dihydropyrimidinone compound of formula (I) wherein X represents O, S, etc. and R′ represents alkyl, alkoxy, thioalkyl, thioalkyloxy, phenyl, substituted phenyl, phenyloxy, substituted phenyloxy, amino, monosubstitutedamino, disubstitutedamino, aryl, heteroaryl, aryloxy, heteroaryloxy, halo; R″ represents alkoxy, phenyloxy, substituted phenyloxy, aryloxy, heteroaryloxy, halo, NR₁R₂ and Rₙ represents OR₁, NH₂, SR₁, NR₁R₂; R₁, R₂=H, alkyl, phenyl, aryl, OCOR₃, SCOR₃, NHCOR₃, NR₁COR₃; R₃ represents alkyl, phenyl, aryl, heteroaryl.

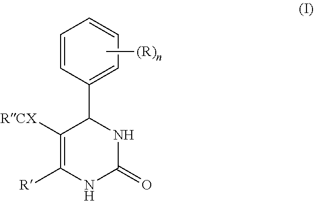

### TECHNICAL FIELD

This invention relates to dihydropyrimidinone compounds for the treatment of cardiovascular diseases and a process for preparing the same.

### BACKGROUND

Drugs that inhibit platelet function have assumed increasing importance in the care of patients with cardiovascular and cerebrovascular disease, which are leading causes of death in the human population.

Physiological systems control fluidity of blood in mammals. Blood must remain fluid in the vascular systems and yet quickly be able to undergo hemostasis. Hemostasis or clotting begins when platelets first adhere to macromolecules in sub-endothelian regions of injured and/or damaged blood vessels. These platelets aggregate to form the primary hemostatic plug and stimulate local activation of plasma coagulation factors leading to generation of a fibrin clot that reinforces aggregated platelets.

Plasma coagulation factors, also referred to as protease zymogens, include factors II, V, VII, VIII, IX, X, XI, and XII. Coagulation or clotting occurs in two ways through different pathways. An intrinsic or contact pathway leads from XII to XIIa to XIa to IXa and to the conversion of X to Xa. Xa with factor Va converts prothrombin (II) to thrombin (IIa) leading to conversion of fibrinogen to fibrin. Polymerization of fibrin leads to a fibrin clot. An extrinsic pathway is initiated by the conversion of coagulation factor VII to VIIa by Xa. Factor VIIa, a plasma protease, is exposed to, and combines with its essential cofactor tissue factor (TF) which resides constitutively beneath the endothelium. The resulting factor VIIa/TF complex proteolytically activates its substrates, factors IX and X, triggering a cascade of reactions that leads to the generation of thrombin and a fibrin clot as described above.

While clotting occurring as a result of an injury to a blood vessel is a critical physiological process for mammals, it can also lead to disease states. A pathological process called thrombosis results when platelet aggregation and/or a fibrin clot blocks (i.e., occludes) a blood vessel. Arterial thrombosis may result in ischemic necrosis of the tissue supplied by the artery. A myocardial infarction or heart attack can result, when thrombosis occurs in a coronary artery. Thrombosis occurring in a vein may cause tissues drained by the vein to become edematous and inflamed. Thrombosis of a deep vein may be complicated by a pulmonary embolism.

Preventing or treating clots in a blood vessel may be therapeutically useful for inhibiting formation of blood platelet aggregates, inhibiting formation of fibrin, inhibiting thrombus formation, inhibiting embolus formation, and for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels.

One such compound is Aspirin. Aspirin inhibits platelet aggregation by irreversible inhibition of platelet cyclooxygenase and thus inhibits the generation of TXA2, a powerful inducer of platelet aggregation and vasoconstriction. Paradoxically, aspirin blocks synthesis of prostacyclin by endothelial cells, resulting in an effect that promotes platelet aggregation.

Clopidogrel hydrogen sulfate is a platelet aggregation inhibitor which was described for the first time in EP 281459. Clopidogrel is a potent, noncompetitive inhibitor of ADP-induced platelet aggregation (Plavix® PI). The active metabolite of clopidogrel binds to the low-affinity ADP-receptors. ADP binding to this site is necessary for activation of the GP IIb/IIIa receptor, which is the binding site for fibrinogen. Fibrinogen links different platelets together to form the platelet aggregate. Clopidogrel thus ultimately inhibits the activation of the GP IIb/IIIa receptor and its binding with fibrinogen.

Dipyridamole has been suggested to act as an antiplatelet drug by several possible mechanisms (Aggrenox® PI). It directly stimulates prostacyclin synthesis, potentiates the platelet inhibitory actions of prostacyclin, and inhibits phosphodiesterase to raise platelet cyclic AMP levels. However, these effects may not occur at therapeutic levels of the drug; hence, the mechanism of action of dipyridamole remains to be elucidated

Other compounds known to exhibit anti-platelet activity include Ticlopidine, Abciximab, Tirofiban, Eptifibatide etc.

Substituted dihydropyrimidinone compounds show interesting biological properties. They have excellent activity against the viruses of the trachoma group. Some of the analogs of Dihydropyrimidine compound's are antitumour agents and found to be active against Walker carcinosarcoma in rats and mice. The cardiovascular activity of Biginelli compounds, namely of P-amino ethyl ester was first discovered by Khanina and co-workers in 1978.

Dihydropyrimidinones have emerged as the integral back-bones of calcium channel blockers (a. Rovnyak, G. C et al, J. Med. Chem., 1995, vol 38, p-119-129; b. Atwal, K. S et al<BR> J. Med. Chem., 1990, vol 33, p-2629-2635), antihypertensive agents (Atwal, K. S et al, J.<BR> <P>Med. Chem., 1991, vol 34, p-806-811), a-adrenergic and neuropeptide Y (NPY) antagonists.

Several marine alkaloids containing the dihydropyrimidine core unit have been known to possess biological activity (a. Overman L. E et at J. Am. Chem. Soc., 1995, vol 117, p-<BR> 2657-2658; b. Snider, B. B et al J. Org. Chem., 1993, vol 58, p-3828-3839). Batzelladine alkaloids have been found to be potent HIV gp-120-CD4 inhibitors (a. Snider, B. B et al <BR> <BR> Tetrahedron Lett., 1996, vol 37, p-6977-6980; b. Patil, A. D et al J. Org. Chem., 1995, vol 60, p-1182-1188). In addition, these compounds exhibit a broad range of biological activities (Kappe, C. O Tetrahedron, 1993, vol 49, p-6937-6963.) such as antiviral, antitumor, antibacterial and anti-inflammatory properties.

Dihrydopyrimidinone compounds were first syntesized by Pietro Beginelli. The of compounds were known as Biginelli compounds. The process comprised reacting numerous aldehydes with urea and a .beta.-keto ester to give a tetrahydropyrimidinone. The Biginelli reaction has been studied, improved upon and a mechanism of formation of tetrahydropyrimidinone proposed. [K. Folkers and T. B. Johnson, J. Am. Chem. Soc., 55, 3784 (1933); J. D. Fissekis, and F. Sweet, J. Am. Chem. Soc., 95, 8741 (1973). The synthesis of dihydropyrimidinones was most often effected using .beta.-keto ester, aryl aldehyde and urea following the principles of Folkers' method, i.e., catalytic amount of acid (e.g., HCl, H.sub.2 SO.sub.4) in protic solvents (e.g., MeOH, EtOH, AcOH) and heating to reflux for a few hours. [K. Folkers and T. B. Johnson, supra]. The method was however associated with several disadvantages. Firstly, the process produced low yields. Secondly, HPLC assays often indicate that a substantive portion of the .beta.-keto ester and aryl aldehyde starting materials is consumed to form alkylidene side product. Thirdly, in cases where acetic acid is used as the solvent system, large amounts of aqueous bases are needed to work up the reaction and the use of sodium bicarbonate or sodium carbonate solutions result in violent bubbling. Alternative methods were subsequently developed which employed a multi-step process to improve the yield of dihyropyrimidinones. (See e.g., K. S. Atwal and B. C. O'Reilly, Heterocycles, 26 (5), 1185 (1987); H. Cho et al., J. Org. Chem., 50, 4227 (1985)].

The inventors of the present invention have found that certain dihydropyrimidinones have been found very effective in the inhibition of ADP induced platelet aggregation. Nitric oxide is known to mediate a number of pharmacological actions such as vasorelaxation, lowering of blood pressure and inhibition of platelet aggregation. The compounds of the present invention have been found to enhance intracellular nitric oxide levels and hence act as antiplatelet agents. The specific acyl group attached with dihydropyrimidinone derivative is attributed for enhancement of intracellular nitric oxide level leading to the inhibition of ADP induced platelet aggregation.

The compounds in accordance with the present invention do not require metabolic activation like Clopidogrel. Moreover, Clopidogrel is reported to have an interaction with other drugs such as atrovastatin and exhibits inter individual variations. The compound of the present invention was found to exhibit significantly higher antiplatelet activity than clopidogrel at equimolar dose exvivo. The compounds of the present invention are effective in causing the inhibition of ADP induced as well as collagen induced platelet aggregation both invitro and exvivo. It is also found to be more potent in inhibition of ADP induced platelet aggregation as compared to the other antiplatelet drug like Aspirin exvivo. The manufacture of these compounds is more cost-effective and economical. As an effective antiplatelet agent, this compound is expected to be useful in the treatment of cardiovascular diseases.

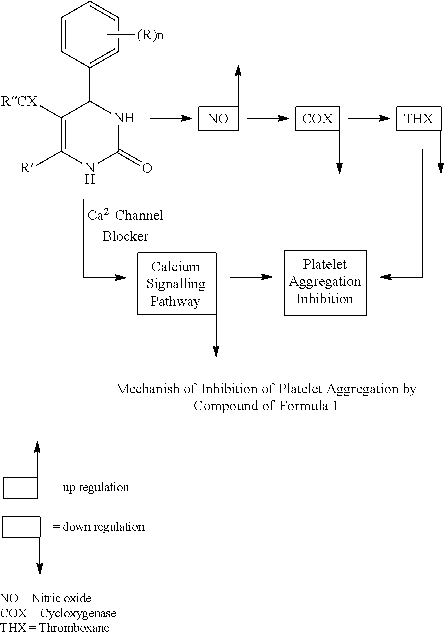

### OBJECTIVE

The principal object of the present invention is to provide dihydropyrimidinone compounds of Formula 1 which act as inhibitors of platelet aggregation.

Another object of the present invention is to provide dihydropyrimidinone compounds of formula 1 which are cost effective and have a better efficacy.

### SUMMARY

The present invention relates to compounds of formula 1

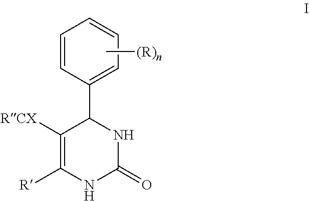

wherein X represents O, S, etc. and R′ represents alkyl, alkoxy, thioalkyl, thioalkyloxy, phenyl, substituted phenyl, phenyloxy, substituted phenyloxy, amino, monosubstitutedamino, disubstitutedamino, aryl, heteroaryl, aryloxy, heteroaryloxy, halo, etc.;
R″ represents alkoxy, phenyloxy, substituted phenyloxy, aryloxy, heteroaryloxy, halo, NR₁R₂, etc. and
Rₙ represents one or several OR₁, NH₂, SR₁, NR₁R₂ wherein R₁, R₂=H, alkyl, phenyl, aryl, OCOR₃, SCOR₃, NHCOR₃, NR₁COR₃,.
wherein R₃ represents alkyl, phenyl, aryl, heteroaryl,.

The present invention further relates to a process of preparing the compound of formula 1 comprising
mixing hydroxyaldehydes, urea, ethylacetoacetate and ferric chloride.hexahydrate in the ratio of 1:3:1.1:0.5.
adding silica gel in the ratio 1:50 with respect to hydroxyaldehydes to the above mixture.
irradiating the resultant mixture obtained for 1-2 mins till the reaction was completed to obtain the product.
purifying the product by column chromatography on silica gel using a gradient solvent system of chloroform-methanol to obtain the pure compound with 80-90% yield.

### DESCRIPTION

The present invention relates to a compound of formula 1 and a process for preparing the same.

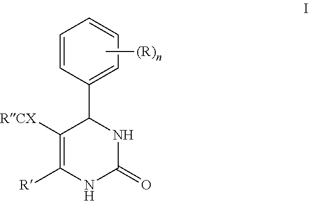

wherein X represents O, S, etc. and R′ represents alkyl, alkoxy, thioalkyl, thioalkyloxy, phenyl, substituted phenyl, phenyloxy, substituted phenyloxy, amino, monosubstitutedamino, disubstitutedamino, aryl, heteroaryl, aryloxy, heteroaryloxy, halo, etc.;
R″ represents alkoxy, phenyloxy, substituted phenyloxy, aryloxy, heteroaryloxy, halo, NR₁R₂, etc. and
Rₙ represents one or several OR_{'}, NH₂, SR₁, NR₁R₂ wherein R₁, R₂=H, alkyl, phenyl, aryl, OCOR₃, SCOR₃, NHCOR₃, NR₁COR₃. wherein R₃ represents alkyl, phenyl, aryl, heteroaryl,.

The specific acyl group attached with dihydropyrimidinone derivative is attributed for enhancement of intracellular nitric oxide level leading to the inhibition of ADP induced platelet aggregation.

The preparation of the compound is carried out by mixing hydroxyaldehydes, urea, ethylacetoacetate and ferric chloride.hexahydrate. Silica gel was then added to the above mixture. The resultant mixture obtained was irradiated with microwave for 1-2 mins till the reaction was completed. The crude product was then purified by column chromatography on silica gel using a gradient solvent system of chloroform-methanol to obtain the pure compound with 80-90% yield. The compound was characterized on the basis of their spectral data analysis.

Synthesis of Dihydropyrimidinones

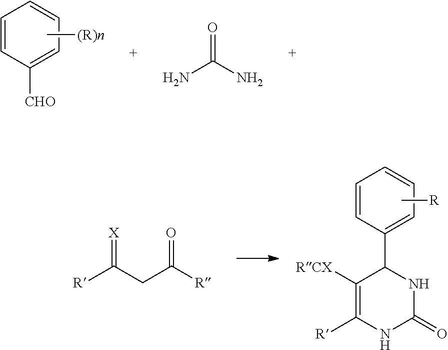

wherein X represents O, S, etc. and R′ represents alkyl, alkoxy, thioalkyl, thioalkyloxy, phenyl, substituted phenyl, phenyloxy, substituted phenyloxy, amino, monosubstitutedamino, disubstitutedamino, aryl, heteroaryl, aryloxy, heteroaryloxy, halo, etc.;
R″ represents alkoxy, phenyloxy, substituted phenyloxy, aryloxy, heteroaryloxy, halo, NR₁R₂, etc. and
Rₙ represents one or several OR₁, NH₂, SR₁, NR₁R₂ wherein R₁, R₂=H, alkyl, phenyl, aryl, OCOR₃, SCOR₃, NHCOR₃, NR₁COR₃.
wherein R₃ represents alkyl, phenyl, aryl, heteroaryl,.

Comparison of the Efficacy of the Compound of Formula 1 With Other Known Antiplatelet Agents

| | | ADP-induced | ADP-induced | |
|---|---|---|---|---|
| | | Platelet | Platelet | |
| | TAase | aggregation (% | aggregation | Enhancement of |
| | Activity | inhibition) | (% inhibition) | Nitric Oxide |
| Structure | (Units) | IN VITRO | EX VIVO | (folds) |
| 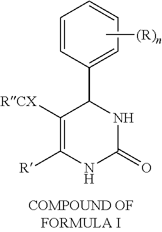 | Nil | 75 | 80 | 8 |
| 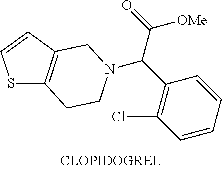 | Nil | Nil | 65 | Nil |
| 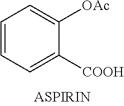 | Nil | Nil | 55 | Nil |

Drugs were suspended in water, sonicated and administered by a gavage.
The test compounds were administered equimolar dose (40 μM).
The concentration of ADP was 15 μM.

The present invention will now be described with the foregoing examples.

### EXAMPLES

1. Preparation of Alkyl/Aryl 4-(Hydroxyaryl)-6-Alkyl/Aryl/Halo-1,2,3,4-Tetrahydropyrimidin-2-One-5-Carboxylates

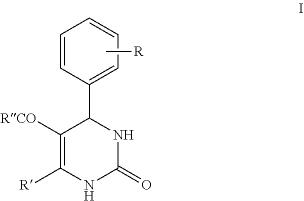

R=OH/NH2/SH/NH-alkyl/NH-aryl, etc
R′=alkyl, phenyl, substituted phenyl, halo, etc
R″=alkoxy, phenyloxy, substituted phenyloxy, halo, etc

To a mixture of hydroxyaldehydes (1 mmol), urea (3 mmol), ethylacetoacetate (1.1 mmol) and ferric chloride.hexahydrate (0.5 mmol), silica gel (5 g) was added to make a thick paste. The resulting mixture was irradiated in a domestic microwave for 1-2 min until TLC showed completion of reaction. The crude product was purified by column chromatography on silica gel using a gradient solvent system of chloroform-methanol to obtain the pure alkyl/aryl 4-(hydroxyaryl)-1,2,3,4-tetrahydropyrimidin-2-one-5-carboxylates in 80-90% yields. The 1,2,3,4-tetrahydropyrimidin-2-ones were characterized on the basis of their spectral data analysis.

2. Preparation of Alkyl/Aryl 4-(Acyloxyaryl)-6-Alkyl/Aryl/Halide-1,2,3,4-Tetrahydropyrimidin-2-One-5-Carboxylates

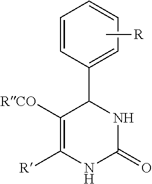

R=O/S/NH/N-alkyl/N-aryl-acyl, O/S/NH/N-alkyl/N-aryl-benzoyl,

O/S/NH/N-alkyl/N-aryl-substituted benzoyl, etc

R′=alkyl, phenyl, substituted phenyl, halo, etc

R″=alkoxy, phenyloxy, substituted phenyloxy, halo, etc

To a solution of alkyl/aryl 4-(hydroxyaryl)-6-alkyl/aryl/halo-1,2,3,4-tetrahydropyrimidin-2-one-5-carboxylates (1 mmol) in acetic anhydride (5 mmol), a catalytic amount of 4-N,N-dimethylaminopyridine was added and the reaction mixture stirred at 25-30° C. for 1 h. The reaction was worked-up by addition of ice-cold water and the aqueous reaction mixture was filtered to afford the corresponding alkyl/aryl 4-(acyloxyaryl)-6-alkyl/aryl/halo-1,2,3,4-tetrahydropyrimidin-2-one-5-carboxylates in quantitative yields.

Preparation of Platelet Rich Plasma for the IN VITRO Studies (7)

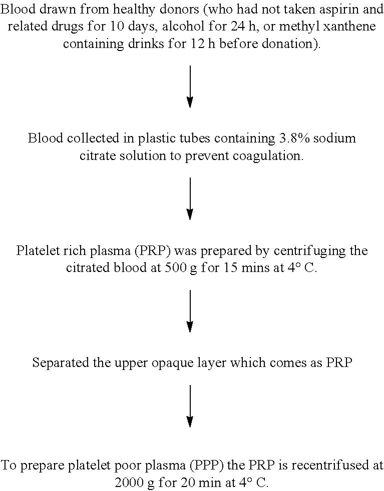

Aggregation Studies

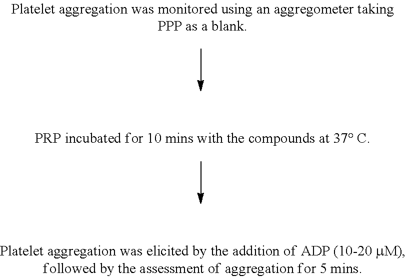

C. Preparation of Platelet Rich Plasma for the EX VIVO Studies

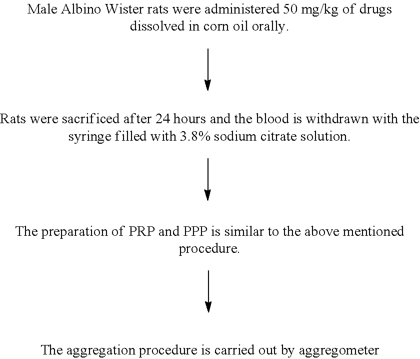

We claim,

1. A dihydropyrimidinone compound of formula I 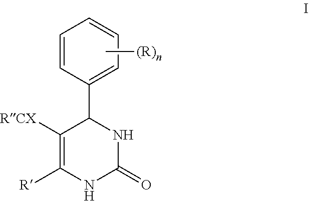
wherein X represents O, S, etc. and R′ represents alkyl, alkoxy, thioalkyl, thioalkyloxy, phenyl, substituted phenyl, phenyloxy, substituted phenyloxy, amino, monosubstitutedamino, disubstitutedamino, aryl, heteroaryl, aryloxy, heteroaryloxy, halo, etc.;
R″ represents alkoxy, phenyloxy, substituted phenyloxy, aryloxy, heteroaryloxy, halo, NR₁R₂, etc. and
Rₙ represents OR₁, NH₂, SR₁, NR₁R₂
R₁, R₂=H, alkyl, phenyl, aryl, OCOR₃, SCOR₃, NHCOR₃, NR₁COR₃.
R₃ represents alkyl, phenyl, aryl, heteroaryl.

2. The dihydropyrimidinone compound as claimed in claim 1, wherein R is OH, OCO-alkyl, OCO-aryl or O-alkyl.

3. The dihydropyrimidinone compound as claimed in claim 1, wherein R′ is alkyl, aryl.

4. A process for preparation of compound of formula I comprising the steps of:
mixing hydroxyaldehyde, urea, ethylacetoacetate and ferric chloride.hexahydrate in the ratio of 1:3:1.1:0.5.
adding silica gel in the ratio 1:50 with respect to hydroxyaldehydes to the above mixture.
irradiating the resultant mixture obtained for 1-2 mins till the reaction was completed to obtain the product.
purifying the product by column chromatography on silica gel using a gradient solvent system of chloroform-methanol to obtain the pure compound with 80-90% yield.

5. The dihydropyrimidinone compound as claimed in claim 1, for use in the inhibition of platelet aggregation.

6. The dihydropyrimidinone compound as claimed in claim 1, for the treatment of cardiovascular diseases.

7. A pharmaceutical formulation comprising therapeutically effective amount of the compound as claimed in claim 1 and any pharmaceutical excipient thereof.

8. The dihydropyrimidinone compound substantially as herein described with reference to foregoing examples.

9. The process for the preparation of the dihydropyrimidinone compound substantially as herein described with reference to foregoing examples.
